SEQUENCE LISTING (2 sequences)

SEQ ID NO: 1 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 2 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

(19) United States
(12) Patent Application Publication
(10) Pub. No.: US 2012/0294875 A1
(43) Pub. Date: Nov. 22, 2012

(54) NON-IMMUNOGLOBULIN ANTIGEN BINDING SCAFFOLDS FOR INHIBITING ANGIOGENESIS AND TUMOR GROWTH

(75) Inventor: Parkash Gill, Agoura Hills, CA (US)

(73) Assignee: VASGENE THERAPEUTICS, INC., Agoura Hills, CA (US)

(21) Appl. No.: 13/439,000

(22) Filed: Apr. 4, 2012

Related U.S. Application Data
(63) Continuation of application No. 12/664,657, filed on Sep. 8, 2010, now abandoned.
(60) Provisional application No. 60/934,767, filed on Jun. 15, 2007.

(51) Int. Cl. C07K 14/435; A61P 35/00; A61P 35/02; A61K 39/00

(52) U.S. Cl. 424/184.1; 530/350

(57) ABSTRACT

In certain embodiments, this present invention provides polypeptide or nucleotide non-immunoglobulin antigen binding scaffold compositions, and methods for inhibiting Ephrin B2 or EphB4 activity. In other embodiments, the present invention provides methods and compositions for treating cancer or for treating angiogenesis-associated diseases.

### RELATED APPLICATIONS

This application claims the benefit of priority of U.S. Provisional Application No. 60/934,767 filed Jun. 15, 2007. The entire teachings of the referenced application are incorporated herein by reference in their entirety.

### BACKGROUND OF THE INVENTION

Angiogenesis, the development of new blood vessels from the endothelium of a preexisting vasculature, is a critical process in the growth, progression, and metastasis of solid tumors within the host. During physiologically normal angiogenesis, the autocrine, paracrine, and amphicrine interactions of the vascular endothelium with its surrounding stromal components are tightly regulated both spatially and temporally. Additionally, the levels and activities of proangiogenic and angiostatic cytokines and growth factors are maintained in balance. In contrast, the pathological angiogenesis necessary for active tumor growth is sustained and persistent, representing a dysregulation of the normal angiogenic system. Solid and hematopoietic tumor types are particularly associated with a high level of abnormal angiogenesis.

It is generally thought that the development of a tumor consists of sequential, and interrelated steps that lead to the generation of an autonomous clone with aggressive growth potential. These steps include sustained growth and unlimited self-renewal. Cell populations in a tumor are generally characterized by growth signal self-sufficiency, decreased sensitivity to growth suppressive signals, and resistance to apoptosis. Genetic or cytogenetic events that initiate aberrant growth sustain cells in a prolonged “ready” state by preventing apoptosis.

It is a goal of the present disclosure to provide agents and therapeutic treatments for inhibiting angiogenesis and tumor growth.

### SUMMARY OF THE INVENTION

In certain aspects, the disclosure provides polypeptide or nucleic acid agents that inhibit EphB4 or EphrinB2 mediated functions, including monomeric ligand binding portions of the EphB4 and EphrinB2 proteins that bind to and affect EphB4 or EphrinB2 in particular ways. As demonstrated herein, EphB4 and EphrinB2 participate in various disease states, including cancers and diseases related to unwanted or excessive angiogenesis. Accordingly, certain polypeptide or nucleic scaffold agents disclosed herein may be used to treat such diseases. In further aspects, the disclosure relates to the discovery that EphB4 and/or EphrinB2 are expressed, often at high levels, in a variety of tumors. Therefore, scaffold agents that downregulate EphB4 or EphrinB2 function may affect tumors by a direct effect on the tumor cells as well as an indirect effect on the angiogenic processes recruited by the tumor. In certain embodiments, the disclosure provides the identity of tumor types particularly suited to treatment with an agent that downregulates EphB4 or EphrinB2 function.

In certain aspects, the disclosure provides antagonist non-immunoglobulin antigen binding scaffolds with an antigen binding domain specific to EphB4 (SEQ ID NO: 1) or ephrin B2 (SEQ ID NO: 2). A non-immunoglobulin antigen binding scaffold may be designed to bind to an extracellular domain of an EphB4 protein and inhibit an activity of the EphB4. A non-immunoglobulin antigen binding scaffold may be designed to bind to an extracellular domain of an Ephrin B2 protein and inhibit an activity of the Ephrin B2. A non-immunoglobulin antigen binding scaffold may be designed to inhibit the interaction between Ephrin B2 and EphB4. An antagonist non-immunoglobulin antigen binding scaffold will generally affect Eph and/or Ephrin signaling. For example, a non-immunoglobulin antigen binding scaffold may inhibit clustering or phosphorylation of Ephrin B2 or EphB4. In some embodiment an antagonist non-immunoglobulin antigen binding scaffold may be essentially any polypeptide comprising a non-immunoglobulin antigen binding scaffold, including, single chain antibodies, diabodies, minibodies, etc.

In certain aspects, the disclosure provides pharmaceutical formulations comprising a non-immunoglobulin antigen binding scaffold reagent and a pharmaceutically acceptable carrier. The non-immunoglobulin antigen binding scaffold reagent may be any disclosed herein. Additional formulations include cosmetic compositions and diagnostic kits.

In certain aspects the disclosure provides methods of inhibiting signaling through Ephrin B2/EphB4 pathway in a cell. A method may comprise contacting the cell with an effective amount of (a) a non-immunoglobulin antigen binding scaffold which binds to an extracellular domain of an EphB4 protein and inhibits an activity of the EphB4; or (b) a non-immunoglobulin antigen binding scaffold which binds to an extracellular domain of an Ephrin B2 protein and inhibits an activity of the Ephrin B2.

In certain aspects the disclosure provides methods for reducing the growth rate of a tumor, comprising administering an amount of a scaffold agent sufficient to reduce the growth rate of the tumor, wherein the scaffold agent is selected from the group consisting of: (a) a non-immunoglobulin antigen binding scaffold which binds to an extracellular domain of an EphB4 protein and inhibits an activity of the EphB4; and (b) a non-immunoglobulin antigen binding scaffold which binds to an extracellular domain of an Ephrin B2 protein and inhibits an activity of the Ephrin B2. Optionally, the tumor comprises cells expressing a higher level of EphB4 and/or EphrinB2 than noncancerous cells of a comparable tissue.

In certain aspects, the disclosure provides methods for treating a patient suffering from a cancer. A method may comprise administering to the patient a scaffold agent selected from the group consisting of: (a) a non-immunoglobulin antigen binding scaffold which binds to an extracellular domain of an EphB4 protein and inhibits an activity of the EphB4; and (b) a non-immunoglobulin antigen binding scaffold which binds to an extracellular domain of an Ephrin B2 protein and inhibits an activity of the Ephrin B2. Optionally, the cancer comprises cancer cells expressing EphrinB2 and/or EphB4 at a higher level than noncancerous cells of a comparable tissue. The cancer may be a metastatic cancer. The cancer may be selected from the group consisting of colon carcinoma, breast tumor, mesothelioma, prostate tumor, squamous cell carcinoma, Kaposi sarcoma, and leukemia. Optionally, the cancer is an angiogenesis-dependent cancer or an angiogenesis independent cancer. The scaffold agent employed may inhibit clustering or phosphorylation of Ephrin B2 or EphB4. A polypeptide agent may be co-administered with one or more additional anti-cancer chemotherapeutic agents that inhibit cancer cells in an additive or synergistic manner with the scaffold agent.

In certain aspects, the disclosure provides methods of inhibiting angiogenesis. A method may comprise contacting a cell with an amount of a scaffold agent sufficient to inhibit angiogenesis, wherein the scaffold agent is selected from the group consisting of: (a) a non-immunoglobulin antigen binding scaffold which binds to an extracellular domain of an EphB4 protein and inhibits an activity of the EphB4; and (b) a non-immunoglobulin antigen binding scaffold which binds to an extracellular domain of an Ephrin B2 protein and inhibits an activity of the Ephrin B2.

In certain aspects, the disclosure provides methods for treating a patient suffering from an angiogenesis-associated disease, comprising administering to the patient a soluble scaffold agent selected from the group consisting of: (a) a non-immunoglobulin antigen binding scaffold which binds to an extracellular domain of an EphB4 protein and inhibits an activity of the EphB4; and (b) a non-immunoglobulin antigen binding scaffold which binds to an extracellular domain of an Ephrin B2 protein and inhibits an activity of the Ephrin B2. The soluble scaffold may be formulated with a pharmaceutically acceptable carrier. An angiogenesis related disease or unwanted angiogenesis related process may be selected from the group consisting of angiogenesis-dependent cancer, benign tumors, inflammatory disorders, chronic articular rheumatism and psoriasis, ocular angiogenic diseases, Osler-Webber Syndrome, myocardial angiogenesis, plaque neovascularization, telangiectasia, hemophiliac joints, angiofibroma, wound granulation, wound healing, telangiectasia psoriasis scleroderma, pyogenic granuloma, cororany collaterals, ischemic limb angiogenesis, rubeosis, arthritis, diabetic neovascularization, fractures, vasculogenesis, and hematopoiesis. A scaffold agent may be co-administered with at least one additional anti-angiogenesis agent that inhibits angiogenesis in an additive or synergistic manner with the soluble scaffold.

In certain aspects, the disclosure provides for the use of a polypeptide or nucleotide scaffold agent in the manufacture of medicament for the treatment of cancer or an angiogenesis related disorder, wherein the scaffold agent is selected from the group consisting of: (a) a non-immunoglobulin antigen binding scaffold which binds to an extracellular domain of an EphB4 protein and inhibits an activity of the EphB4; and (b) a non-immunoglobulin antigen binding scaffold which binds to an extracellular domain of an Ephrin B2 protein and inhibits an activity of the Ephrin B2.

In certain aspects, the disclosure provides methods for treating a patient suffering from a cancer, comprising: (a) identifying in the patient a tumor having a plurality of cancer cells that express EphB4 and/or EphrinB2; and (b) administering to the patient a scaffold agent selected from the group consisting of: (i) a non-immunoglobulin antigen binding scaffold which binds to an extracellular domain of an EphB4 protein and inhibits an activity of the EphB4; and (ii) a non-immunoglobulin antigen binding scaffold which binds to an extracellular domain of an Ephrin B2 protein and inhibits an activity of the Ephrin B2. Optionally, a method may comprise identifying in the patient a tumor having a plurality of cancer cells having a gene amplification of the EphB4 and/or EphrinB2 gene.

In certain aspects, the disclosure provides scaffold agents that inhibit EphB4 or Ephrin 132 mediated functions, including non-immunoglobulin antigen binding scaffolds and antigen binding portions thereof that bind to and affect EphB4 in particular ways. As demonstrated herein, EphB4 and EphrinB2 participate in various disease states, including cancers and diseases related to unwanted or excessive angiogenesis. Accordingly, certain scaffold agents disclosed herein may be used to treat such diseases. In further aspects, the disclosure relates to the discovery that EphB4 and/or EphrinB2 are expressed, often at high levels, in a variety of tumors. Therefore, scaffold agents that downregulate EphB4 or EphrinB2 function may affect tumors by a direct effect on the tumor cells as well as an indirect effect on the angiogenic processes recruited by the tumor. In certain embodiments, the disclosure provides the identity of tumor types particularly suited to treatment with an agent that downregulates EphB4 or EphrinB2 function.

In certain aspects, the disclosure provides an isolated non-immunoglobulin antigen binding scaffold comprising an antigen binding domain that binds to an epitope situated in the extracellular portion of EphB4 and inhibits an EphB4 activity. The isolated non-immunoglobulin antigen binding scaffold comprising an antigen binding domain may bind to an epitope situated within amino acids 16-198 of the EphB4 sequence. For example, the epitope may be situated within the Globular Domain (GD) of EphB4 that binds to EphrinB2. The isolated non-immunoglobulin antigen binding scaffold comprising an antigen binding domain may inhibit the binding of EphB4 to the extracellular portion of EphrinB2. The isolated non-immunoglobulin antigen binding scaffold comprising an antigen binding domain may bind to an epitope situated within amino acids 327-427 or 428-537 of the EphB4 sequence. For example, the isolated non-immunoglobulin antigen binding scaffold comprising an antigen binding domain may bind to the first fibronectin-like domain (FND1) or the second fibronectin-like domain (FND2) of EphB4. The isolated non-immunoglobulin antigen binding scaffold comprising an antigen binding domain may inhibit EphB4 dimerization or multimerization and may optionally inhibit the EphrinB2-stimulated autophosphorylation of EphB4. The isolated non-immunoglobulin antigen binding scaffold comprising an antigen binding domain may inhibit the formation of tubes by cultured endothelial cells, the vascularization of a tissue in vivo, the vascularization of tissue implanted in the cornea of an animal, the vascularization of a Matrigel tissue plug implanted in an animal, and/or the growth of a human tumor xenograft in a mouse. Preferred non-immunoglobulin antigen binding scaffolds that bind to an epitope situated within amino acids 16-198 of the EphB4 sequence. Preferred non-immunoglobulin antigen binding scaffolds that bind to an epitope situated within amino acids 428-537 of the EphB4 sequence.

In certain aspects, the disclosure provides a non-immunoglobulin antigen binding scaffold comprising an antigen binding domain that binds to an epitope situated in the extracellular portion of EphB4 and stimulates EphB4 kinase activity. For example, described herein are isolated non-immunoglobulin antigen binding scaffolds or antigen binding portion thereof that bind to an epitope situated within amino acids 327-427 of the EphB4 sequence and stimulate EphB4 kinase activity. The isolated non-immunoglobulin antigen binding scaffold comprising an antigen binding domain may bind to the first fibronectin-like domain (FND1) of EphB4.

In certain aspects, the disclosure provides antagonist non-immunoglobulin antigen binding scaffolds with an antigen binding domain specific to EphB4 and ephrin B2. A non-immunoglobulin antigen binding scaffold may be designed to bind to an extracellular domain of an EphB4 protein and inhibit an activity of the EphB4. A non-immunoglobulin antigen binding scaffold may be designed to bind to an extracellular domain of an Ephrin B2 protein and inhibit an activity of the Ephrin B2. A non-immunoglobulin antigen binding scaffold may be designed to inhibit the interaction between Ephrin B2 and EphB4. In certain embodiments, the non-immunoglobulin antigen binding scaffold comprising an antigen binding domain prevents antibody binding to an epitope of EphB4 or Ephrin B2.

The disclosure provides a method of treating cancer, the method comprising administering to a patient in need thereof an effective amount of an isolated non-immunoglobulin antigen binding scaffold comprising an antigen binding domain that binds to an epitope situated in the extracellular portion of EphB4 or Ephrin B2 and either inhibits an EphB4 or Ephrin B2 activity or activates EphB4 or Ephrin B2 kinase activity. Optionally the patient has been diagnosed with a cancer selected from the group consisting of colon carcinoma, breast tumor, mesothelioma, prostate tumor, squamous cell carcinoma, Kaposi sarcoma, and leukemia. The isolated non-immunoglobulin antigen binding scaffold comprising an antigen binding domain may be administered systemically or locally. Additionally, the disclosure provides methods of inhibiting angiogenesis in a patient, the method comprising administering to a patient in need thereof an effective amount of an isolated non-immunoglobulin antigen binding scaffold comprising an antigen binding domain that binds to an epitope situated in the extracellular portion of EphB4 or Ephrin B2 and inhibits an EphB4 or Ephrin B2 activity or activates an EphB4 or Ephrin B2 kinase activity. Optionally, the patient is diagnosed with macular degeneration.

In certain aspects, the disclosure provides a pharmaceutical preparation comprising any of the isolated non-immunoglobulin antigen binding scaffolds or antigen binding portions thereof disclosed herein, as well as the use of such non-immunoglobulin antigen binding scaffolds or antigen binding portions thereof to make a pharmaceutical preparation for treating cancer. Optionally, the cancer is selected from the group consisting of colon carcinoma, breast tumor, mesothelioma, prostate tumor, squamous cell carcinoma, Kaposi sarcoma, and leukemia.

In certain aspects, the non-immunoglobulin antigen binding scaffolds disclosed herein may be covalently linked (or otherwise stably associated with) an additional functional moiety, such as a label or a moiety that confers desirable pharmacokinetic properties. Exemplary labels include those that are suitable for detection by a method selected from the group consisting of: fluorescence detection methods, positron emission tomography detection methods and nuclear magnetic resonance detection methods. Labels may, for example, be selected from the group consisting of: a fluorescent label, a radioactive label, and a label having a distinctive nuclear magnetic resonance signature. Moieties such as a polyethylene glycol (PEG) moiety may be affixed to a non-immunoglobulin antigen binding scaffold comprising an antigen binding domain to increase serum half-life.

In certain aspects, the non-immunoglobulin antigen binding scaffolds disclosed herein may be derived from a reference protein by having a mutated amino acid sequence. The non-immunoglobulin antigen binding scaffold may be derived from an antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class 1 MHC, T-cell antigen receptor, CD1, C2 and 1-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

### DEFINITIONS

By “non-immunoglobulin antigen binding scaffold” is meant an antibody mimic or antibody-like scaffold. Non-immunoglobulin antigen binding scaffolds of the application may contain an immunoglobulin-like fold. Examples of such non-immunoglobulin antigen binding scaffold include: antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class 1 MHC, T-cell antigen receptor, CD1, C2 and 1-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

By “immunoglobulin-like fold” is meant a domain of between about 80-150 amino acid residues that includes two layers of antiparallel beta-sheets, and in which the flat, hydrophobic faces of the two beta-sheets are packed against each other. Proteins according to the invention may include several immunoglobulin-like folds covalently bound or associated non-covalently into larger structures.

By “scaffold.” is meant a framework which can specifically bind to a target. Scaffolds may be composed of amino acids or nucleotides but are not limited to these embodiments.

### DETAILED DESCRIPTION OF THE INVENTION

### 1. Overview

The current invention is based in part on the discovery that signaling through the ephrin/ephrin receptor (ephrin/eph) pathway contributes to tumorigenesis. Applicants detected expression of EphB4 and ephrin B2 in tumor tissues and developed anti-tumor therapeutic agents for blocking signaling through the ephrin/eph (see U.S. Patent Application numbers: 20050249736 and 20050084873). This disclosure provides non-immunoglobulin antigen binding scaffold therapeutic agents and methods for non-immunoglobulin antigen binding scaffold-based inhibition of the function of EphB4 and/or Ephrin B2. Accordingly, in certain aspects, the disclosure provides numerous polypeptide and nucleotide scaffolds (agents) that may be used to treat cancer as well as angiogenesis related disorders and unwanted angiogenesis related processes.

As used herein, the terms Ephrin and Eph are used to refer, respectively, to ligands and receptors. They can be from any of a variety of animals (e.g., mammals/non-mammals, vertebrates/non-vertebrates, including humans). The nomenclature in this area has changed rapidly and the terminology used herein is that proposed as a result of work by the Eph Nomenclature Committee, which can be accessed, along with previously-used names at web site http://www.eph-nomenclature.com.

The work described herein, refers to Ephrin B2 and EphB4. However, the present invention contemplates any ephrin ligand and/or Eph receptor within their respective family, which is expressed in a tumor. The ephrins (ligands) are of two structural types, which can be further subdivided on the basis of sequence relationships and, functionally, on the basis of the preferential binding they exhibit for two corresponding receptor subgroups. Structurally, there are two types of ephrins: those which are membrane-anchored by a glycerophosphatidylinositol (GP1) linkage and those anchored through a transmembrane domain. Conventionally, the ligands are divided into the Ephrin-A subclass, which are GP1-linked proteins which bind preferentially to EphA receptors, and the Ephrin-B subclass, which are transmembrane proteins which generally bind preferentially to EphB receptors.

The Eph family receptors are a family of receptor protein-tyrosine kinases which are related to Eph, a receptor named for its expression in an erythropoietin-producing human hepatocellular carcinoma cell line. They are divided into two subgroups on the basis of the relatedness of their extracellular domain sequences and their ability to bind preferentially to Ephrin-A proteins or Ephrin-B proteins. Receptors which interact preferentially with Ephrin-A proteins are EphA receptors and those which interact preferentially with Ephrin-B proteins are EphB receptors.

Eph receptors have an extracellular domain composed of the ligand-binding globular domain, a cysteine rich region followed by a pair of fibronectin type III repeats. The cytoplasmic domain consists of a juxtamembrane region containing two conserved tyrosine residues; a protein tyrosine kinase domain; a sterile α-motif (SAM) and a PDZ-domain binding motif EphB4 is specific for the membrane-bound ligand Ephrin B2 (Sakano, S. et al 1996; Brambilla R. et al 1995). Ephrin B2 belongs to the class of Eph ligands that have a transmembrane domain and cytoplasmic region with five conserved tyrosine residues and PDZ domain. Eph receptors are activated by binding of clustered, membrane attached ephrins, indicating that contact between cells expressing the receptors and cells expressing the ligands is required for Eph activation.

Upon ligand binding, an Eph receptor dimerizes and autophosphorylate the juxtamembrane tyrosine residues to acquire full activation. In addition to forward signaling through the Eph receptor, reverse signaling can occur through the ephrin Bs. Eph engagement of ephrins results in rapid phosphorylation of the conserved intracellular tyrosines and somewhat slower recruitment of PDZ binding proteins. Recently, several studies have shown that high expression of Eph/ephrins may be associated with increased potentials for tumor growth, tumorigenicity, and metastasis.

In certain embodiments, the present invention provides non-immunoglobulin antigen binding scaffolds or antibody mimics that inhibit activity of Ephrin B2, EphB4, or both. For example, such polypeptide or nucleotide therapeutic agents can inhibit the function of Ephrin B2 or EphB4, inhibit the interaction between Ephrin B2 and EphB4, inhibit the phosphorylation of Ephrin B2 or EphB4, or inhibit any of the downstream signaling events upon binding of Ephrin B2 to EphB4.

In the immune system, specific Abs are selected and amplified from a large library (affinity maturation). The processes can be reproduced in vitro using combinatorial library technologies. The successful display of Ab fragments on the surface of bacteriophage has made it possible to generate and screen a vast number of CDR mutations. An increasing number of Fabs and Fvs (and their derivatives) is produced by this technique, providing a rich source for structural studies. The combinatorial technique can be combined with Ab mimics.

A number of protein domains that could potentially serve as protein scaffolds have been expressed as fusions with phage capsid proteins. Review in Clackson & Wells, Trends Biotechnol. 12:173-184 (1994). Indeed, several of these protein domains have already been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L, ed.) pp. 517-524, Academic Press, San Diego (1994)). These scaffolds have displayed a single randomized loop or region.

An advantage of antibody mimics over antibody fragments is structural. These antibody mimics are derived from whole, stable, and soluble structural scaffolds. For example, the Fn3 scaffold is found in the human body. Consequently, they exhibit better folding and thermostability properties than antibody fragments, whose creation involves the removal of parts of the antibody native fold, often exposing amino acid residues that, in an intact antibody, would be buried in a hydrophobic environment, such as an interface between variable and constant domains. Exposure of such hydrophobic residues to solvent increases the likelihood of aggregation of the antibody fragments.

In the case of protein scaffolds a protein is used to select or design a protein framework which can specifically bind to a target. When designing proteins from the scaffold, amino acid residues that are important for the framework's favorable properties are retained, while others residues may be varied. Such a scaffold has less than 50% of the amino acid residues that vary between protein derivatives having different properties and greater than or equal to 50% of the residues that are constant between such derivatives. Most commonly, these constant residues confer the same overall three-dimensional fold to all the variant domains, regardless of their properties.

### II. Non-Immunoglobulin Antigen Binding Scaffolds

In one embodiment, non-immunoglobulin antigen binding scaffolds of the invention are specific for the extracellular portion of the Ephrin B2 or EphB4 protein. In another embodiment, non-immunoglobulin antigen binding scaffolds of the invention are specific for the intracellular portion or the transmembrane portion of the Ephrin B2 or EphB4 protein. In a further embodiment, non-immunoglobulin antigen binding scaffolds of the invention are specific for the extracellular portion of the Ephrin B2 or EphB4 protein.

The EphB4 and Ephrin B2 scaffolds described herein may be used to treat a variety of disorders, particularly cancers and disorders related to unwanted angiogenesis. The disclosure provides non-immunoglobulin antigen binding scaffolds and antigen binding portions thereof that inhibit one or more EphB4 or Ephrin B2 mediated functions, such as EphrinB2 or Eph B4 binding; or EphB4 or Ephrin B2 kinase activity. Such binding agents may be used to inhibit EphB4 or Ephrin B2 function in vitro and in vivo, and preferably for treating cancer or disorders associated with unwanted angiogenesis.

EphB4 belongs to a family of transmembrane receptor protein tyrosine kinases. The extracellular portion of EphB4 is composed of the ligand-binding domain (also referred to as globular domain), a cysteine-rich domain, and a pair of fibronectin type III repeats. The cytoplasmic domain consists of a juxtamembrane region containing two conserved tyrosine residues; a protein tyrosine kinase domain; a sterile α-motif (SAM) and a PDZ-domain binding motif. EphB4 is specific for the membrane-bound ligand Ephrin B2. EphB4 is activated by binding of clustered, membrane-attached ephrin ligands, indicating that contact, between cells expressing the receptor and cells expressing the ligand, is required for the Eph receptor activation. Upon ligand binding, an EphB4 receptor dimerizes and autophosphorylates the juxtamembrane tyrosine residues to acquire full activation.

As used herein, the term EphB4 refers to an EphB4 polypeptide from a mammal including humans. In one embodiment, the non-immunoglobulin antigen binding scaffolds are designed against an isolated and/or recombinant mammalian EphB4 or portion thereof (e.g., peptide) or against a host cell which expresses recombinant mammalian EphB4. In certain aspects, non-immunoglobulin antigen binding scaffolds of the invention specifically bind to an extracellular domain of an EphB4 protein (referred to herein as an anti-EphB4 soluble scaffold). As used herein, the anti-EphB4 soluble scaffolds include fragments, functional variants, and modified forms of anti-EphB4 soluble scaffolds.

In certain aspects, non-immunoglobulin antigen binding scaffolds of the invention specifically bind to an extracellular domain (ECD) of an EphB4 protein (also referred to herein as a soluble anti-EphB4 scaffold). A soluble anti-EphB4 scaffold may comprise a sequence encompassing the globular (G) domain (amino acids 29-197 of SEQ ID NO: 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). As used herein, the anti-EphB4 soluble scaffolds include fragments, functional variants, and modified forms of anti-EphB4 soluble scaffolds.

In certain aspects, the present invention provides non-immunoglobulin antigen binding scaffolds (anti-EphB4 or Ephrin B2) having binding specificity for an EphB4 or Ephrin B2; or a portion of EphB4 or Ephrin B2. Optionally, the immunoglobulins can bind to EphB4 or Ephrin B2 with an affinity of at least about 1×10⁻⁶, 1−10⁻⁷, 1×10⁻⁸, 1×10⁻⁹ M or less. Optionally, non-immunoglobulin antigen binding scaffolds and portions thereof bind to EphrinB2 or EphB4 with an affinity that is roughly equivalent to that of a soluble extracellular EphB4 or Ephrin B2 polypeptide comprising the globular ligand binding domain. Non-immunoglobulin antigen binding scaffolds disclosed herein will preferably be specific for EphB4 or Ephrin B2, with minimal binding to other members of the Eph or Ephrin families.

In certain embodiments, non-immunoglobulin antigen binding scaffolds of the present invention bind to one or more specific domains of EphB4. For example, a non-immunoglobulin antigen binding scaffold binds to one or more extracellular domains of EphB4 (such as the globular domain, the cystein-rich domain, and the first fibronectin type 3 domain, and the second fibronectin type 3 domain). Optionally, the subject non-immunoglobulin antigen binding scaffold may bind to at least two domains of an EphB4.

In addition, functional fragments of non-immunoglobulin antigen binding scaffolds can also be produced. Functional fragments of the subject non-immunoglobulin antigen binding scaffolds retain at least one binding function and/or modulation function of the full-length non-immunoglobulin antigen binding scaffold from which they are derived. Preferred functional fragments retain an antigen binding function of a corresponding full-length non-immunoglobulin antigen binding scaffold (e.g., specificity for an EphB4 or Ephrin B2). Certain preferred functional fragments retain the ability to inhibit one or more functions characteristic of an EphB4 or Ephrin B2, such as a binding activity, a signaling activity, and/or stimulation of a cellular response. For example, in one embodiment, a functional fragment of an EphB4 or Ephrin B2 non-immunoglobulin antigen binding scaffold can inhibit the interaction of EphB4 or Ephrin B2 with one or more of its ligands or receptors (e.g., Ephrin B2 or EphB4) and/or can inhibit one or more receptor-mediated functions, such as cell migration, cell proliferation, angiogenesis, and/or tumor growth.

In certain embodiments, the present invention provides EphB4 or Ephrin B2 antagonist non-immunoglobulin antigen binding scaffolds. As described herein, the term “antagonist non-immunoglobulin antigen binding scaffold” refers to a non-immunoglobulin antigen binding scaffold that can inhibit one or more functions of an EphB4 or Ephrin B2, such as a binding activity (e.g., ligand binding) and a signaling activity (e.g., clustering or phosphorylation of EphB4 or Ephrin B2, stimulation of a cellular response, such as stimulation of cell migration or cell proliferation). For example, an antagonist non-immunoglobulin antigen binding scaffold can inhibit (reduce or prevent) the interaction of an EphB4 or Ephrin B2 receptor with a natural ligand or receptor (e.g., Ephrin B2 or EphB4 or fragments thereof). Preferably, antagonist non-immunoglobulin antigen binding scaffolds directed against EphB4 or Ephrin B2 can inhibit functions mediated by EphB4 or Ephrin B2, including endothelial cell migration, cell proliferation, angiogenesis, and/or rumor growth. Optionally, the antagonist non-immunoglobulin antigen binding scaffold binds to an extracellular domain of EphB4 or Ephrin B2.

In other embodiments, the present invention provides EphB4 kinase activating non-immunoglobulin antigen binding scaffolds. Such non-immunoglobulin antigen binding scaffolds enhance EphB4 kinase activity, even independent of EphrinB2. In some instances, such non-immunoglobulin antigen binding scaffolds may be used to stimulate EphB4. However, applicants note that in most cell-based and in vivo assays, activating antibodies of EphB4 surprisingly behaved like antagonist antibodies (as shown in examples 1-13 in U.S. Patent Application number: 20050249736). Such antibodies appear to bind to the fibronectin type III domains, particularly the region of amino acids 327-427. While not wishing to be limited to any particular mechanism, it is expected that these non-immunoglobulin antigen binding scaffolds stimulate not only EphB4 kinase activity, but also EphB4 removal from the membrane, thus decreasing overall EphB4 levels.

In certain embodiments, the present invention provides EphrinB2 kinase activating non-immunoglobulin antigen binding scaffolds. Such non-immunoglobulin antigen binding scaffolds enhance EphrinB2 kinase activity, even independent of EphB4. In some instances, such non-immunoglobulin antigen binding scaffolds may be used to stimulate EphrinB2.

In certain embodiments, the non-immunoglobulin antigen binding scaffolds are further attached to a label that is able to be detected (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A binding agent affixed to such a moiety may be used as an imaging agent and is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography. Immunoscintigraphy using non-immunoglobulin antigen binding scaffolds or other binding polypeptides directed at EphB4 or Ephrin B2 may be used to detect and/or diagnose cancers and vasculature. For example, non-immunoglobulin antigen binding scaffolds against the EphB4 or Ephrin B2 marker labeled with. ⁹⁹Technetium, ¹¹¹Indium, ¹²⁵Iodine-may be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

The antibody mimics described herein may be fused to other protein domains. For example, these mimics may be integrated with the human immune response by fusing the constant region of an IgG (F_{c}) with an antibody mimic, such as a ¹⁰Fn3 module (the tenth Fn3 module of human fibronectin), preferably through the C-terminus of ¹⁰Fn3. The Fc in such a ¹⁰Fn3-Fc fusion molecule activates the complement component of the immune response and increases the therapeutic value of the antibody mimic. Similarly, a fusion between an antibody mimic, such as ¹⁰Fn3, and a complement protein, such as Clq, may be used to target cells, and a fusion between an antibody mimic, such as ¹⁰Fn3, and a toxin may be used to specifically destroy cells that carry a particular antigen. In addition, a non-immunoglobulin antigen binding scaffold, such as ¹⁰Fn3, in any form may be fused with albumin to increase its half-life in the bloodstream and its tissue penetration. Any of these fusions may be generated by standard techniques, for example, by expression of the fusion protein from a recombinant fusion gene constructed using publically available gene sequences.

In addition to monomers, any of the scaffold constructs described herein may be generated as dimers or multimers of antibody mimics as a means to increase the valency and thus the avidity of antigen binding. Such multimers may be generated through covalent binding. For example, individual ¹⁰Fn3 modules may be bound by imitating the natural ¹⁰Fn3-Fn3-¹⁰Fn3 C-to-N-terminus binding or by imitating antibody dimers that are held together through their constant regions. A ¹⁰Fn3-Fc construct may be exploited to design dimers of the general scheme of ¹⁰Fn3-Fc::Fc-¹⁰Fn3. The bonds engineered into the Fc::Fc interface may be covalent or non-covalent. In addition, dimerizing or multimerizing partners other than Fc can be used in hybrids, such as ¹⁰Fn3 hybrids, to create such higher order structures.

In particular examples, covalently bonded multimers may be generated by constructing fusion genes that encode the multimer or, alternatively, by engineering codons for cysteine residues into monomer sequences and allowing disulfide bond formation to occur between the expression products. Non-covalently bonded multimers may also be generated by a variety of techniques. These include the introduction, into monomer sequences, of codons corresponding to positively and/or negatively charged residues and allowing interactions between these residues in the expression products (and therefore between the monomers) to occur. This approach may be simplified by taking advantage of charged residues naturally present in a monomer subunit, for example, the negatively charged residues of fibronectin. Another means for generating non-covalently bonded antibody mimics is to introduce, into the monomer gene (for example, at the amino- or carboxy-termini), the coding sequences for proteins or protein domains known to interact. Such proteins or protein domains include coil-coil motifs, leucine zipper motifs, and any of the numerous protein subunits (or fragments thereof) known to direct formation of dimers or higher order multimers.

Many constrained protein scaffolds, capable of presenting a protein of interest as a conformationally-restricted domain have been identified, including minibody structures (Bianchi et al. (1994) J Mol Biol 236:649-659), loops on β-sheet turns, coiled-coil stem structures (Myszka & Chaiken (1994) Biochem 33:2363-2372), zinc-finger domains, cysteine-linked (disulfide) structures, transglutaminase linked structures, cyclic peptides, helical barrels or bundles, leucine zipper motifs (Martin et al. (1994) EMBO J 13:5303-5309), etc. (see Skerra, J Mol Recognit. 2000 July-August; 13(4):167-87). The following examples of scaffolds of the disclosure are not intended to be limiting.

### Antibody Substructures

Functional substructures of Abs can be prepared by proteolysis and by recombinant methods. They include the Fab fragment, which contains the VH-CH1 domains of the heavy chain and the VL-CL1 domains of the light chain joined by a single interchain disulfide bond, and the Fv fragment, which contains only the VH and VL domains. In some cases, a single VH domain retains significant affinity (Ward et al., Nature 341:544-546 (1989)). It has also been shown that a certain monomeric κlight chain will specifically bind to its cognate antigen. Separated light or heavy chains have sometimes been found to retain some antigen-binding activity (Ward et al., Nature 341:544-546 (1989)). These antibody fragments are not suitable for structural analysis using NMR spectroscopy due to their size, low solubility or low conformational stability.

Another functional substructure is a single chain Fv (scFv), made of the variable regions of the immunoglobulin heavy and light chain, covalently connected by a peptide linker. These small (Mᵣ 25,000) proteins generally retain specificity and affinity for antigen in a single polypeptide and can provide a convenient building block for larger, antigen-specific molecules. Several groups have reported biodistribution studies in xenografted athymic mice using scFv reactive against a variety of tumor antigens, in which specific tumor localization has been observed. However, the short persistence of scFvs in the circulation limits the exposure of tumor cells to the scFvs, placing limits on the level of uptake. As a result, tumor uptake by scFvs in animal studies has generally been only 1-5% ID/g as opposed to intact antibodies that can localize in tumors ad 30-40% ID/g and have reached levels as high as 60-70% ID/g.

A small protein scaffold called a “minibody” was designed using a part of the Ig VH domain as the template (Pessi et al., Nature. 1993 Mar. 25; 362(6418):367-9). Minibodies with high affinity (dissociation constant (K_{d}) about 10⁻⁷ M) to interleukin-6 were identified by randomizing loops corresponding to CDR1 and CDR2 of VH and then selecting mutants using the phage display method. These experiments demonstrated that the essence of the Ab function could be transferred to a smaller system. However, the minibody had inherited the limited solubility of the VH domain.

It has been reported that camels (Camelus dromedarius) often lack variable light chain domains when IgG-like material from their serum is analyzed, suggesting that sufficient antibody specificity and affinity can be derived form VH domains (three CDR loops) alone. Davies and Riechmann recently demonstrated that “camelized” VH domains with high affinity (K K_{d} about 10⁻⁷ M) and high specificity can be generated by randomizing only the CDR3. To improve the solubility and suppress nonspecific binding, three mutations were introduced to the framework region (Davies & Riechmann, Protein Eng. 1996 June; 9(6):531-7). It has not been definitively shown, however, that camelization can be used, in general, to improve the solubility and stability of VHs.

An alternative to the “minibody” is the “diabody.” Diabodies are small bivalent and bispecific antibody fragments, i.e., they have two antigen-binding sites. The fragments contain a heavy-chain variable domain (V_{H}) connected to a light-chain variable domain (V_{L}) on the same polypeptide chain (V_{H}-V_{L}). Diabodies are similar in size to an Fab fragment. By using a linker that is too short to allow pairing between the two domains on the same chain, the domains are forced to pair with the complementary domains of another chain and create two antigen-binding sites. These dimeric antibody fragments, or “diabodies,” are bivalent and bispecific.

Since the development of the monoclonal antibody technology, a large number of 3D structures of Ab fragments in the complexed and/or free states have been solved by X-ray crystallography. Analysis of Ab structures has revealed that five out of the six CDRs have limited numbers of peptide backbone conformations, thereby permitting one to predict the backbone conformation of CDRs using the so-called canonical structures. The analysis also has revealed that the CDR3 of the VH domain (VH-CDR3) usually has the largest contact surface and that its conformation is too diverse for canonical structures to be defined; VH-CDR3 is also known to have a large variation in length. Therefore, the structures of crucial regions of the Ab-antigen interface still need to be experimentally determined.

Comparison of crystal structures between the free and complexed states has revealed several types of conformational rearrangements. They include side-chain rearrangements, segmental movements, large rearrangements of VH-CDR3 and changes in the relative position of the VH and VL domains. In the free state, CDRs, in particular those which undergo large conformational changes upon binding, are expected to be flexible. Since X-ray crystallography is not suited for characterizing flexible parts of molecules, structural studies in the solution state have not been possible to provide dynamic pictures of the conformation of antigen-binding sites.

Antibody mimics of the disclosure may also be CDR peptides. CDR peptides and organic CDR mimetics have been successfully designed (Dougall et al., Trends Biotechnol. 1994 September; 12(9):372-9). CDR peptides are short, typically cyclic, peptides which correspond to the amino acid sequences of CDR loops of antibodies. CDR loops are responsible for antibody-antigen interactions. Organic CDR mimetics are peptides corresponding to CDR loops which are attached to a scaffold, e.g., a small organic compound.

CDR peptides and organic CDR mimetics have been shown to retain some binding affinity. However, as expected, they are too small and too flexible to maintain full affinity and specificity. Mouse CDRs have been grafted onto the human Ig framework without the loss of affinity, though this “humanization” does not solve the above-mentioned problems specific to solution studies.

The non-immunoglobulin antigen binding scaffolds of this disclosure may be domain antibodies. Domain Antibodies (dAbs) are small functional binding units of antibodies, corresponding to the variable regions of either the heavy (V_{H}) or light (V_{L}) chains of human antibodies. Domain Antibodies have a molecular weight of approximately 13 kDa, or less than one-tenth the size of a full antibody (see U.S. Patent Application number: 20040202995).

The non-immunoglobulin antigen binding scaffolds of this disclosure may be small modular immunopharmaceutical (SMIP™, Trubion) drugs (see U.S. Patent Application number: 20050175614). These biologics are binding domain-immunoglobulin fusion proteins that feature a binding domain for a cognate structure such as an antigen, a counterreceptor or the like, a hinge region polypeptide having either zero or one cysteine residue, and immunoglobulin CH2 and CH3 domains, and that are capable of ADCC and/or CDC while occurring predominantly as monomeric polypeptides. These single-chain polypeptides are engineered for full binding and activity function of a monoclonal antibody (mAb). Approximately one-third to one-half the size of conventional therapeutic mAbs, SMIP drugs maintain in vivo half-life and high expression levels.

### Fibronectin-Like Molecules

The non-immunoglobulin antigen binding scaffolds of this disclosure may be Adnectins (Koide et al., J Mol Biol. 1998 December 11; 284(4):1141-51). The fibronectin type III domain (FN3) is a small autonomous folding unit which occurs in many animal proteins involved in ligand binding. The beta-sandwich structure of FN3 closely resembles that of immunoglobulin domains. In exemplary embodiments, the FN3 domain is the ¹⁰Fn3 module (the tenth Fn3 module of human fibronectin).

Although ¹⁰Fn3 represents a preferred scaffold for the generation of antibody mimics, other molecules may be substituted for ¹⁰Fn3 in the molecules described herein. These include, without limitation, human fibronectin modules ¹⁰Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary scaffolds having immunoglobulin-like folds include N-cadherin, ICAM-2, titin, GCSF receptor, cytokine receptor, glycosiclase inhibitor, E-cadherin, and antibiotic chromoprotein. Alternatively, any other protein that includes one or more immunoglobulin-like folds may be utilized. Such proteins may be identified, for example, using the program SCOP (Murzin et al., J. Mol. Biol. 247:536 (1995); Lo Conte et al., Nucleic Acids Res. 25:257 (2000).

Generally, any molecule that exhibits a structural relatedness to the VH domain may be utilized as an antibody mimic. Such molecules may, like fibronectin, include three loops at the N-terminal pole of the molecule and three loops at the C-terminal pole, each of which may be randomized to create diverse libraries; alternatively, larger domains may be utilized, having larger numbers of loops, as long as a number of such surface randomizable loops are positioned closely enough in space that they can participate in antigen binding. Examples include T-cell antigen receptor and superoxide dismutase, which each have four loops that can be randomized; and an Fn3 dimer, tissue factor domains, and cytokine receptor domains, each of which have three sets of two similar domains where three randomizable loops are part of the two domains (bringing the total number of loops to six).

In yet another alternative, any protein having variable loops positioned close enough in space may be utilized for candidate binding protein production. For example, large proteins having spatially related, solvent accessible loops may be used, even if unrelated structurally to an immunoglobulin-like fold. Exemplary proteins include, without limitation, cytochrome F, green fluorescent protein, GroEL, and thaumatin. The loops displayed by these proteins may be randomized and superior binders selected from a randomized library as described herein. Because of their size, molecules may be obtained that exhibit an antigen binding surface considerably larger than that found in an antibody-antigen interaction. Other useful scaffolds of this type may also be identified using the program SCOP (Murzin et al., J. Mol. Biol. 247:536 (1995)) to browse among candidate proteins having numerous loops, particularly loops positioned among parallel beta sheets or a number of alpha-helices.

Modules from different organisms and parent proteins may be most appropriate for different applications. For example, in designing an antibody mimic, it may be most desirable to generate that protein from a fibronectin or fibronectin-like molecule native to the organism for which a therapeutic is intended. In contrast, the organism of origin is less important or even irrelevant for antibody mimics that are to be used for in vitro applications, such as diagnostics, or as research reagents. For any of these molecules, libraries may be generated and used to select binding proteins by any of the methods described herein.

### Anticalins

The non-immunoglobulin antigen binding scaffolds of this disclosure may be anticalins, lipocalin derivatives (see U.S. Patent Application number: 20060058510). The lipocalins (Pervaiz and Brew, FASEB J. 1 (1987), 209-214) are a family of small, often monomeric secretory proteins which have been isolated from various organisms, and whose physiological role lies in the storage or in the transport of different ligands as well as in more complex biological functions (Flower, Biochem. J. 318 (1996), 1-14). The lipocalins bear relatively little mutual sequence similarity and their belonging to the same protein structural family was first eluicidated by X-ray structure analysis (Sawyer et al., Nature 327 (1987), 659).

The first lipocalin of known spatial structure was the retinol-binding protein, Rbp, which effects the transport of water-insoluble vitamin A in blood serum (Newcomer et al., EMBO J. 3 (1984), 1451-1454). Shortly thereafter the tertiary structure of the bilin-binding protein, Bbp, from the butterfly Picris brassicae was determined (Huber et al., J. Mol. Biol. 195 (1987), 423-434). The essential structural features of this class of proteins can be illustrated with the help of the spatial structure of this lipocalin. The central element in the folding architecture of the lipocalins is the cylindrical β-pleated sheet structure, the so-called β-barrel, which is made up of eight nearly circularly arranged antiparallel β-strands.

This supersecondary structural element can also be viewed as a “sandwich”-arrangement of two four-stranded β-sheet structures. Additional structural elements are an extended segment at the amino-terminus of the polypeptide chain and an α-helix close to the carboxy-terminus, which itself is followed by an extended segment. These additional features are, however, not necessarily revealed in all lipocalins. For example a significant part of the N-terminal segment is missing in the epididymal retinoic acid-binding protein (Newcomer, Structure 1 (1993), 7-18). Additional peculiar structural elements are also known, such as for example membrane anchors (Bishop and Weiner, Trends Biochem. Sci. 21 (1996), 127) which are only present in certain lipocalins.

The β-barrel is closed on one end by dense amino acid packing as well as by loop segments. On the other end the β-barrel forms a binding pocket in which the respective ligand of the lipocalin is complexed. The eight neighbouring antiparallel β-strands there are connected in a respective pairwise fashion by hairpin bends in the polypeptide chain which, together with the adjacent amino acids which are still partially located in the region of the cylindrical β-pleated sheet structure, each form a loop element. The binding pocket for the ligands is formed by these in total four peptide loops. In the case of Bbp, biliverdin Iχγ is complexed in this binding pocket. Another typical ligand for lipocalins is vitamin A in the case of Rbp as well as β-lactoglobulin (Papiz et al., Nature 324 (1986), 383-385).

Alignments of the sequences from different representatives of the lipocalin family can be found in, among other publications, the publication by Cowan et al. (Proteins: Struct., Funct., Genet. 8 (1990), 44-61) and in the review article by Flower (FEBS Lett. 354 (1994), 7-11). Among the currently many more than 20 different known lipocalins, there exist mainly two human proteins which have already been biochemically characterized in detail: the retinol-binding protein and the apolipoprotein D, ApoD (Yang et al., Biochemistry 33 (1994), 12451-12455). ApoD is especially interesting since it bears a close structural relationship with the Bbp mentioned above (Peitsch and Boguski, New Biologist 2 (1990), 197-206).

### Natural and Artificial Helix Bundle Proteins

The non-immunoglobulin antigen binding scaffolds of this disclosure may be affibodies (U.S. Patent numbers: 5831012, 6534628 and 6740734; and Gunneriusson et al., Protein Eng. 1999 October; 12(10):873-8). Affibodies are novel proteins obtainable by mutagenesis of surface-exposed amino acids of domains of natural bacterial receptors said proteins being obtained without substantial loss of basic structure and stability of said natural bacterial receptors. Said proteins have preferably been selected from a protein library embodying a repertoire of said novel proteins. In such novel bacterial receptor structures, at least one amino acid residue involved in the interaction fuction of the original bacterial receptor has been made subject to substitution by another amino acid residue so as to result in substantial loss of the original interaction capacity with a modified interaction capacity being created, said substitution being made without substantial loss of basic structure and stability of the original bacterial receptor.

It is preferred that said bacterial structures originate from Gram-positive bacteria. Among such bacteria there may be mentioned Staphylococcus aureus, Streptococcus pyogenes [group A], Streptococcus group C, G, L, bovine group G streptococci, Streptococcus zooepidemicus [group C], Streptococcus zooepidemicus S212, Streptococcus pyogenes, streptococci groups A, C, G, Peptostreptococcus magnus, Streptococcus agalactiae. Of special interest are thermophilic bacteria evolved to persist in environments of elevated temperatures. Receptors from species like e.g. Bacillus stearothermophilus, Thermus aquaticus, Thermococcus litoralis and Pyrococcus have the potential of being naturally exceptionally stable, thus suitable for providing structural frameworks for protein engineering according to the invention.

It is particularly preferred to use as a starting material for the modification of the interaction function bacterial receptor structures originating from staphylococcal protein A or streptococcal protein G. Among preferred receptors there may be mentioned bacterial receptors originating from Fc[IgG]receptor type I, type II, type III, type IV, type V and type VI, fibronectin receptor, M protein, plasmin receptor, collagen receptor, fibrinogen receptor or protein L [K light chains], protein H [human IgG], protein B [Human IgA, A1], protein Arp [human IgA]. Particularly preferred bacterial receptors originate from the Fc[IgG]receptor type I of staphylococcal protein A or the serum albumin receptor of streptococcal protein G.

In order to maintain stability and the properties of the original bacterial receptor structure it is preferred in accordance with the present invention that the substitution involving amino acid residues taking part in the interaction function of the original bacterial receptor does not involve more than about 50% of the amino acid residues of the original bacterial receptor. It is particularly preferred that not more than about 25% of the amino acid residues of the original bacterial receptor are made subject to substitution.

In regard to the original bacterial receptor structures selected from modification of their interaction functions it is particularly preferred to use receptors originating from the IgG-binding domains Z, Cl, and the serum albumin binding domains B2A3. In order to maintain as far as possible the stability and properties of the original receptor structure subject to modification in accordance with the present invention it is preferred that substitution thereof involves not more than substantially all of the amino acid residues taking part in the interaction function of the original bacterial receptor.

In order to obtain favourable properties concerning stability and resistance to various conditions it is preferred that the bacterial receptor according to the present invention is comprised of not more than about 100 amino acid residues. It is known from scientific reports that proteins of a relatively small size are fairly resistant to increased temperatures and also to low pH and certain chemicals. For details concerning temperature resistance c.f. the article by Alexander et al. in Biochemistry 1992, 31, pp 3597-3603. With regard to the modification of the natural bacterial receptor structure it is preferred to perform the substitution thereof by resorting to a genetic engineering, such as site-directed mutagenesis. Affibodies may also be generated by phage display.

The non-immunoglobulin antigen binding scaffolds of this disclosure may be derived derived from additional natural and artificial helix bundle proteins such as: Cytochrome b562 (Ku and Schultz, PNAS 1995 Jul. 3; 92(14):6552-6) and Zinc Finger scaffolds (Handel and DeGrado, Science. 1993 Aug. 13; 261(5123):879-85).

### Knottins

The non-immunoglobulin antigen binding scaffolds of this disclosure may be knottins. The elucidation, in 1982, of the X-ray structure of PCI, a carboxypeptidase inhibitor from potato, revealed for the first time a “knotted” structure in which a disulfide bridge was shown to penetrate a macrocycle formed by two other disulfides and the interconnecting backbone segments (Rees & Lipscomb, J Mol Biol. 1982 Sep. 25; 160(3):475-98.) In 1989, this peculiar scaffold was shown to also appear in the squash trypsin inhibitors, and later on in toxins from cone snails and spiders. This structural family now extends to 12 different families and more than 80 experimentally determined structures. This structural family is referred to as knottins (Le Nguyen et al., Biochimie. 1990 June-July; 72(6-7):431-5), although other names are also used (i.e. Inhibitor Cystine Knots or ICK. The specific interest in this particular scaffold has come from the observation that these proteins are very small, and thus readily accessible to chemical synthesis, yet remarkably stable thanks to the high content in disulfide bridges and the “knotted” topology.

### Enzyme Active Sites

The non-immunoglobulin antigen binding scaffolds of this disclosure may be glubodies. The immunoglobulin framework has been mutagenized to engineer recombinant libraries of proteins as potential diagnostics and novel catalysts, although the often shallow binding cleft may limit the utility of this framework for binding diverse small organic molecules. By contrast, the glutathione S-transferase (GST) family of enzymes contains a deep binding cleft, which has evolved to accommodate a broad range of hydrophobic xenobiotics. GST molecules with novel ligand-binding characteristics may be produced by random mutagenesis of segments of the binding cleft. There are two ligand-recognition segments (LRSs) in human GST P1, which are near the active site in the folded protein, not essential for the overall structure or activity of the protein. Libraries of GST P1-derived proteins may be produced by substituting randomized sequences for an LRS or inserting random sequences into an LRS. The recombinant proteins in the libraries, collectively designated as ‘glubodies,’ generally retain enzymatic activity but differ markedly both from each other and from the parent enzyme in sensitivity to inhibition by diverse small organic compounds. In some instances, a glubody is inhibited by completely novel structures.

Human glutathione transferase A1-1 can be expressed as a fusion protein with coat protein III of filamentous phage f1 in a form that allows selection among variant mutant forms based on specific adsorption to immobilized active-site ligands. Novel glutathione transferases with altered specificity for active-site ligands can be isolated by adsorption of the fusion protein on the surface of phage to analogs of an electrophilic substrate (Widersten and Mannervik, J Mol Biol. 1995 Jul. 7; 250(2):115-22). Thus, phage display of glutathione transferase affords a system for engineering novel binding specificities onto the pre-existing protein framework of the enzyme.

The non-immunoglobulin antigen binding scaffolds of this disclosure may be derived from thioredoxin. Another enzyme that uses glutathione (GSH) as a co-substrate, E. coli thioredoxin (TrxA), has been employed as a scaffold for the display of single conformationally constrained peptides replacing its active site (Colas et al., Nature. 1996 April 11; 380(6574):548-50). TrxA is a small cytosolic enzyme normally involved in maintaining the thiol/disulphide equilibrium inside the cell. It is highly soluble, rigid, can be overexpressed in large amounts, and its three-dimensional structure is known.

### Triose Phosphate Isomerase (TIM)

The non-immunoglobulin antigen binding scaffolds of this disclosure may be derived from triose phosphate isomerase. Triose phosphate isomerase (TIM) family of enzymes, whose well conserved (a/b)8 barrel obviously represents a preferred scaffold for the creation of biocatalysts during evolution. An attractive property of these enzymes is the bipartite character of the active centre, with the arrangement of substrate-binding residues primarily within the barrel itself and the catalytic residues mostly in the connecting loop regions (Altamirano et al., Nature. 2000 Feb. 10; 403(6770):617-22).

### Staphylococcal Nuclease Scaffold

The non-immunoglobulin antigen binding scaffolds of this disclosure may be derived from staphylococcal nuclease scaffold. A catalytically inactive version of staphylococcal nuclease was employed as a scaffold in order to display a peptamcr library consisting of 16 random amino acids within budding yeast cells, again followed by selection for inhibitors of biological pathways (Norman et al., Science. 1999 Jul. 23; 285(5427):591-5). Staphylococcal nuclease was chosen as a carrier protein because it is small, folds spontaneously in the absence of chaperones, can be produced at high levels in eukaryotes as well as prokaryotes, and has a prominently exposed loop on its surface.

### C-Type Lectin-Like Domain Proteins

The non-immunoglobulin antigen binding scaffolds of this disclosure may be derived from the C-type lectin-like domain proteins (International Patent Application Publication No. WO04039841A2, WO04005335A3, WO0248189A2, WO 98/56906A2, and US Patent Application No. 20040132094). The C-type lectin-like domain (CTLD) is a protein domain family which has been identified in a number of proteins isolated from many animal species. Initially, the CTLD domain was identified as a domain common to the so-called C-type lectins (calcium-dependent carbohydrate binding proteins) and named “Carbohydrate Recognition Domain” (“CRD”). More recently, it has become evident that this domain is shared among many eukaryotic proteins, of which several do not bind sugar moieties, and hence, the canonical domain has been named as CTLD.

CTLDs have been reported to bind a wide diversity of compounds, including carbohydrates, lipids, proteins, and even ice. Only one copy of the CTLD is present in some proteins, whereas other proteins contain from two to multiple copies of the domain. In the physiologically functional unit multiplicity in the number of CTLDs is often achieved by assembling single copy protein protomers into larger structures.

The CTLD consists of approximately 120 amino acid residues and, characteristically, contains two or three intra-chain disulfide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops. Several CTLDs contain either one or two binding sites for calcium and most of the side chains which interact with calcium are located in the loop-region.

On the basis of CTLDs for which 3D structural information is available, it has been inferred that the canonical CTLD is structurally characterised by seven main secondary-structure elements (i.e. five β-strands and two α-helices) sequentially appearing in the order β1; α1; α2; β2; β3; β4; and β5. In all CTLDs, for which 3D structures have been determined, the β-strands are arranged in two anti-parallel β5-sheets, one composed of β31 and β5, the other composed of β2, β3 and β4. An additional β-strand, β0, often precedes β1 in the sequence and, where present, forms an additional strand integrating with the β1, β5-sheet. Further, two disulfide bridges, one connecting α1 and β5 (C₁-C.sub_{IV}) and one connecting β3 and the polypeptide segment connecting β4 and β5 (C_{II}-C_{III}) are invariantly found in all CTLDs characterised so far. In the CTLD 3D-structure, these conserved secondary structure elements form a compact scaffold for a number of loops, which in the present context collectively are referred to as the “loop-region”, protruding out from the core. These loops are in the primary structure of the CTLDs organised in two segments, loop segment A, LSA, and loop segment B, LSB. LSA represents the long polypeptide segment connecting β2 and β3 which often lacks regular secondary structure and contains up to four loops. LSB represents the polypeptide segment connecting the β-strands β3 and β4. Residues in LSA, together with single residues in β4, have been shown to specify the Ca²⁺- and ligand-binding sites of several CTLDs, including that of tetranectin. E.g. mutagenesis studies, involving substitution of single or a few residues, have shown, that changes in binding specificity, Ca²⁺-sensitivity and/or affinity can be accommodated by CTLD domains. One such system, where the protein used as scaffold is tetranectin or the CTLD domain of tetranectin, is envisaged as a system of particular interest, not least because the stability of the trimeric complex of tetranectin protomers is very high.

The non-immunoglobulin antigen binding scaffolds of this disclosure may be tetranectins (so-named plasminogen kringle 4 domain-binding protein). Tetranectin is a trimeric glycoprotein, which has been isolated from human plasma and found to be present in the extra-cellular matrix in certain tissues. Tetranectin is known to bind calcium, complex polysaccharides, plasminogen, fibrinogen/fibrin, and apolipoprotein (a). The interaction with plasminogen and apolipoprotein (a) is mediated by the so-called kringle 4 protein domain therein. This interaction is known to be sensitive to calcium and to derivatives of the amino acid lysine. A human tetranectin gene has been characterised, and both human and murine tetranectin cDNA clones have been isolated. Both the human and the murine mature protein comprise 181 amino acid residues. The 3D-structures of full length recombinant human tetranectin and of the isolated tetranectin CTLD have been determined. Tetranectin is a two- or possibly three-domain protein, i.e. the main part of the polypeptide chain comprises the CTLD (amino acid residues Gly53 to Val181), whereas the region Leu26 to Lys52 encodes an alpha-helix governing trimerisation of the protein via the formation of a homotrimeric parallel coiled coil. The polypeptide segment Glu1 to Glu25 contains the binding site for complex polysaccharides (Lys6 to Lys15) and appears to contribute to stabilisation of the trimeric structure. The two amino acid residues Lys148 and Glu150, localised in loop 4, and Asp165 (localised in β4) have been shown to be of critical importance for plasminogen kringle 4 binding, whereas the residues Ile140 (in loop 3) and Lys166 and Arg167 (in β4) have been shown to be of some importance. Substitution of Thr149 (in loop 4) with an aromatic residue has been shown to significantly increase affinity of tetranectin to kringle 4 and to increase affinity for plasminogen kringle 2 to a level comparable to the affinity of wild type tetranectin for kringle 4.

### Protease Inhibitors

Protease inhibitors are widely known as small and remarkably stable proteins. In most cases their proteasebinding site comprises a short, more or less extended peptide stretch with varying sequence being presented as an exposed loop by a structural framework that is specific for the inhibitor family.

The non-immunoglobulin antigen binding scaffolds of this disclosure may be Kunitz domain proteins. Bovine pancreatic trypsin inhibitor (BPTI) other Kunitz-type serine protease inhibitors have been developed as scaffolds (US. Patent Application No. 20040209243, Roberts et al., Gene. 1992 Nov. 2; 121(1):9-15; Shimomura et al., J. Biol. Chem. 272: 6370-76 (1997)). The protein inhibitors of serine proteases can be classified into at least 10 families, according to various schemes. Among them, serpins, such as maspin (Sheng et al., Proc. Natl. Acad. Sci. USA 93: 11669-74 (1996)) and Kunitz-type inhibitors, such as urinary trypsin inhibitor (Kobayashi et al., Cancer Res. 54: 844-49 (1994)) have been previously implicated in suppression of cancer progression. The Kunitz-type inhibitors form very tight, but reversible complexes with their target serine proteases. The reactive sites of these inhibitors are rigid and can simulate optimal protease substrates. The interaction between a serine protease and a Kunitz-type inhibitor depends on complementary, large surface areas of contact between the protease and inhibitor. The inhibitory activity of the recovered Kunitz-type inhibitor from protease complexes can always be reconstituted. The Kunitz-type inhibitors may be cleaved by cognate proteases, but such cleavage is not essential for their inhibitory activity. In contrast, serpin-type inhibitors also form tight, stable complexes with proteases; in most of cases these complexes are even more stable than those containing Kunitz-type inhibitors. Cleavage of serpins by proteases is necessary for their inhibition, and serpins are always recovered in a cleaved, inactive form from protease reactions.

The non-immunoglobulin antigen binding scaffolds of this disclosure may be derived derived from tendamistat. Researchers have used the small 74 amino acid α-amylase inhibitor Tendamistat as a presentation scaffold on the filamentous phage M13 (McConnell and Hoess, J Mol Biol. 1995 Jul. 21; 250(4):460-70). Tendamistat is a β-sheet protein from Streptomyces tendae. It has a number of features that make it an attractive scaffold for peptides, including its small size, stability, and the availability of high resolution NMR and X-ray structural data. Tendamistat's overall topology is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. By analogy with the CDR loops found in immunoglobulins, the loops the Tendamistat may serve a similar function and can be easily randomized by in vitro mutagenesis.

The non-immunoglobulin antigen binding scaffolds of this disclosure may be derived from other protease inhibitors comprising: pancreatic secretory trypsin inhibitor (PSTI) (Rottgen and Collins, Gene. 1995 Oct. 27; 164(2):243-50); Ecotin (Wang et al., J Biol Chem. 1995 May 19; 270(20):12250-6); and LACI-D1 (Markland et al., Biochemistry. 1996 Jun. 18; 35(24):8045-57).

### Nucleotide Aptamers

Aptamers may be nucleic acid molecules having specific binding affinity to molecules through interactions other than classic Watson-Crick base pairing. Aptamers, like peptides generated by phage display or monoclonal antibodies (MAbs), are capable of specifically binding to selected targets and, through binding, block their targets' ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes (see U.S. Patent Application numbers: 20060084109, 20030064931).

### III. Applications

The non-immunoglobulin antigen binding scaffolds of the present invention are useful in a variety of applications, including research, diagnostic and therapeutic applications. For instance, they can be used to isolate and/or purify receptor or portions thereof, and to study receptor structure (e.g., conformation) and function.

In certain aspects, the various non-immunoglobulin antigen binding scaffolds of the present invention can be used to detect or measure the expression of EphB4 or Ephrin B2, for example, on endothelial cells (e.g., venous endothelial cells), or on cells transfected with an EphB4 or Ephrin B2 gene. Thus, they also have utility in applications such as cell sorting and imaging (e.g., flow cytometry, and fluorescence activated cell sorting), for diagnostic or research purposes.

In certain embodiments, the non-immunoglobulin antigen binding scaffolds or antigen binding fragments of the non-immunoglobulin antigen binding scaffolds can be labeled or unlabeled for diagnostic purposes. Typically, diagnostic assays entail detecting the formation of a complex resulting from the binding of a non-immunoglobulin antigen binding scaffold to EphB4 or Ephrin B2. The non-immunoglobulin antigen binding scaffolds can be directly labeled. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654; and 4,098,876). When unlabeled, the non-immunoglobulin antigen binding scaffolds can be used in assays, such as agglutination assays. Unlabeled non-immunoglobulin antigen binding scaffolds can also be used in combination with another (one or more) suitable reagent which can be used to detect non-immunoglobulin antigen binding scaffold, such as a labeled antibody (e.g., a second antibody) reactive with the non-immunoglobulin antigen binding scaffold or other suitable reagent (e.g., labeled protein A).

In one embodiment, the non-immunoglobulin antigen binding scaffolds of the present invention can be utilized in enzyme immunoassays, wherein the subject non-immunoglobulin antigen binding scaffolds, or second non-immunoglobulin antigen binding scaffolds, are conjugated to an enzyme. When a biological sample comprising an EphB4 or Ephrin B2 protein is combined with the subject non-immunoglobulin antigen binding scaffolds, binding occurs between the non-immunoglobulin antigen binding scaffolds and EphB4 or Ephrin B2 protein. In one embodiment, a sample containing cells expressing an EphB4 or Ephrin B2 protein (e.g., endothelial cells) is combined with the subject non-immunoglobulin antigen binding scaffolds, and binding occurs between the non-immunoglobulin antigen binding scaffolds and cells bearing an EphB4 or Ephrin B2 protein comprising an epitope recognized by the non-immunoglobulin antigen binding scaffold. These bound cells can be separated from unbound reagents and the presence of the non-immunoglobulin antigen binding scaffold-enzyme conjugate specifically bound to the cells can be determined, for example, by contacting the sample with a substrate of the enzyme which produces a color or other detectable change when acted on by, the enzyme. In another embodiment, the subject non-immunoglobulin antigen binding scaffolds can be unlabeled, and a second, labeled antibody can be added which recognizes the non-immunoglobulin antigen binding scaffold.

In certain aspects, kits for use in detecting the presence of an EphB4 or Ephrin B2 protein in a biological sample can also be prepared. Such kits will include a non-immunoglobulin antigen binding scaffold which binds to an EphB4 or Ephrin B2 protein or portion of said receptor, as well as one or more ancillary reagents suitable for detecting the presence of a complex between the non-immunoglobulin antigen binding scaffold and EphB4 or Ephrin B2 or portion thereof. The non-immunoglobulin antigen binding scaffold compositions of the present invention can be provided in lyophilized form, either alone or in combination with additional non-immunoglobulin antigen binding scaffolds specific for other epitopes. The non-immunoglobulin antigen binding scaffolds, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the non-immunoglobulin antigen binding scaffolds can be provided as a lyophilized mixture with the adjunct ingredients, or the adjunct ingredients can be separately provided for combination by the user. Generally these adjunct materials will be present in less than about 5% weight based on the amount of active non-immunoglobulin antigen binding scaffold, and usually will be present in a total amount of at least about 0.001% weight based on non-immunoglobulin antigen binding scaffold concentration. Where a second antibody capable of binding to the non-immunoglobulin antigen binding scaffold is employed, such antibody can be provided in the kit, for instance in a separate vial or container. The second antibody, if present, is typically labeled, and can be formulated in an analogous manner with the antibody formulations described above.

Similarly, the present invention also relates to a method of detecting and/or quantitating expression of an EphB4 or Ephrin B2 or a portion thereof by a cell, wherein a composition comprising a cell or fraction thereof (e.g., membrane fraction) is contacted with a non-immunoglobulin antigen binding scaffold which binds to an EphB4 or Ephrin B2 or a portion thereof under conditions appropriate for binding of the non-immunoglobulin antigen binding scaffold thereto, and non-immunoglobulin antigen binding scaffold binding is monitored. Detection of the non-immunoglobulin antigen binding scaffold, indicative of the formation of a complex between non-immunoglobulin antigen binding scaffold and EphB4 or Ephrin B2 or a portion thereof, indicates the presence of the receptor. Binding of non-immunoglobulin antigen binding scaffold to the cell can be determined by standard methods. The method can be used to detect expression of EphB4 or Ephrin B2 on cells from an individual. Optionally, a quantitative expression of EphB4 or Ephrin B2 on the surface of endothelial cells can be evaluated, for instance, by flow cytometry, and the staining intensity can be correlated with disease susceptibility, progression or risk.

The present disclosure also relates to a method of detecting the susceptibility of a mammal to certain diseases. To illustrate, the method can be used to detect the susceptibility of a mammal to diseases which progress based on the amount of EphB4 or Ephrin B2 present on cells and/or the number of EphB4- or Ephrin B2-positive cells in a mammal. In one embodiment, the invention relates to a method of detecting susceptibility of a mammal to a tumor. In this embodiment, a sample to be tested is contacted with a non-immunoglobulin antigen binding scaffold which binds to an EphB4 or Ephrin B2 or portion thereof under conditions appropriate for binding of said non-immunoglobulin antigen binding scaffold thereto, wherein the sample comprises cells which express EphB4 or Ephrin B2 in normal individuals. The binding of non-immunoglobulin antigen binding scaffold and/or amount of binding is detected, which indicates the susceptibility of the individual to a tumor, wherein higher levels of receptor correlate with increased susceptibility of the individual to a tumor. Applicants and other groups have found that expression of EphB4 or Ephrin B2 has a correlation with tumor growth and progression. The non-immunoglobulin antigen binding scaffolds of the present invention can also be used to further elucidate the correlation of EphB4 or Ephrin B2 expression with progression of angiogenesis-associated diseases in an individual.

The antibody mimics described herein may be used in any technique for evolving new or improved binding proteins. In one particular example, the target of binding is immobilized on a solid support, such as a column resin or microtiter plate well, and the target contacted with a library of candidate scaffold-based binding proteins. Such a library may consist of antibody mimic clones, such as ¹⁰Fn3 clones constructed from the wild type ¹⁰Fn3 scaffold through randomization of the sequence and/or the length of the ¹⁰Fn3 CDR-like loops. If desired, this library may be an RNA-protein fusion library generated, for example, by the techniques described in Szostak et al., U.S. Ser. No. 09/007,005 and 09/247,190; Szostak et al., WO98/31700; and Roberts & Szostak, Proc. Natl. Acad. Sci. USA (1997) vol. 94, p. 12297-12302. Alternatively, it may be a DNA-protein library (for example, as described in Lohse, DNA-Protein Fusions and Uses Thereof, U.S. Ser. No. 60/110,549, U.S. Ser. No. 09/459,190, and WO 00/32823). The fusion library is incubated with the immobilized target, the support is washed to remove non-specific binders, and the tightest binders are eluted under very stringent conditions and subjected to PCR to recover the sequence information or to create a new library of binders which may be used to repeat the selection process, with or without further mutagenesis of the sequence. A number of rounds of selection may be performed until binders of sufficient affinity for the antigen are obtained.

In one particular example, the ¹⁰Fn3 scaffold may be used as the selection target. For example, if a protein is required that binds a specific peptide sequence presented in a ten residue loop, a single ¹⁰Fn3 clone is constructed in which one of its loops has been set to the length of ten and to the desired sequence. The new clone is expressed in vivo and purified, and then immobilized on a solid support. An RNA-protein fusion library based on an appropriate scaffold is then allowed to interact with the support, which is then washed, and desired molecules eluted and re-selected as described above.

Similarly, the scaffolds described herein, for example, the ¹⁰Fn3 scaffold, may be used to find natural proteins that interact with the peptide sequence displayed by the scaffold, for example, in an ¹⁰Fn3 loop. The scaffold protein, such as the ¹⁰Fn3 protein, is immobilized as described above, and an RNA-protein fusion library is screened for binders to the displayed loop. The binders are enriched through multiple rounds of selection and identified by DNA sequencing.

In addition, in the above approaches, although RNA-protein libraries represent exemplary libraries for directed evolution, any type of scaffold-based library may be used in the selection methods of the invention.

### IV. Methods of Treatment

In certain embodiments, the present invention provides methods of inhibiting angiogenesis and methods of treating angiogenesis-associated diseases. In other embodiments, the present invention provides methods of inhibiting or reducing tumor growth and methods of treating an individual suffering from cancer. These methods involve administering to the individual a therapeutically effective amount of one or more scaffold therapeutic agents as described above. These methods are particularly aimed at therapeutic and prophylactic treatments of animals, and more particularly, humans.

As described herein, angiogenesis-associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation and wound healing; telangiectasia psoriasis scleroderma, pyogenic granuloma, cororany collaterals, ischemic limb angiogenesis, corneal diseases, rubeosis, arthritis, diabetic neovascularization, fractures, vasculogenesis, hematopoiesis.

It is understood that methods and compositions of the invention are also useful for treating any angiogenesis-independent cancers (tumors). As used herein, the term “angiogenesis-independent cancer” refers to a cancer (tumor) where there is no or little neovascularization in the tumor tissue.

In particular, scaffold therapeutic agents of the present invention are useful for treating or preventing a cancer (tumor), including, but not limited to, colon carcinoma, breast cancer, mesothelioma, prostate cancer, bladder cancer, squamous cell carcinoma of the head and neck (HNSCC), Kaposi sarcoma, and leukemia.

In certain embodiments of such methods, one or more scaffold therapeutic agents can be administered, together (simultaneously) or at different times (sequentially). In addition, polypeptide therapeutic agents can be administered with another type of compounds for treating cancer or for inhibiting angiogenesis.

In certain embodiments, the subject methods of the invention can be used alone. Alternatively, the subject methods may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject scaffold therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a scaffold therapeutic agent of the present invention is administered in combination with another conventional anti-neoplastic agent, either concomitantly or sequentially, such therapeutic agent is shown to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disrupters such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as an anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6573256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin αᵥβ₃, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of the scaffold therapeutic agents of the invention may be continued while the other therapy is being administered and/or thereafter. Administration of the scaffold therapeutic agents may be made in a single dose, or in multiple doses. In some instances, administration of the scaffold therapeutic agents is commenced at least several days prior to the conventional therapy, while in other instances, administration is begun either immediately before or at the time of the administration of the conventional therapy.

### V. Methods of Administration and Pharmaceutical Compositions

In certain embodiments, the subject non-immunoglobulin antigen binding scaffolds of the present invention are formulated with a pharmaceutically acceptable carrier. Such therapeutic agents can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject scaffold therapeutic agents include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of anti-tumor or anti-angiogenesis therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject scaffold therapeutic agent as an active ingredient.

In solid dosage forms for oral administration (capsules, tablets, pills, dragees, powders, granules, and the like), one or more scaffold therapeutic agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microcmulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention can be administered topically, either to skin or to mucosal membranes such as those on the cervix and vagina. This offers the greatest opportunity for direct delivery to tumor with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject scaffold therapeutic agents may be mixed under sterile conditions with a pharmaceutically acceptable carrier, and with any preservatives, buffers, or propellants which may be required. The ointments, pastes, creams and gels may contain, in addition to a subject scaffold agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject scaffold therapeutic agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more scaffold therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more scaffold therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal or rectally administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In other embodiments, the scaffold therapeutic agents of the instant invention can be expressed within cells from eukaryotic promoters. For example, a non-immunoglobulin antigen binding scaffold can be expressed in eukaryotic cells from an appropriate vector. The vectors are preferably DNA plasmids or viral vectors. Viral vectors can be constructed based on, but not limited to, adeno-associated virus, retrovirus, adenovirus, or alphavirus. Preferably, the vectors stably introduced in and persist in target cells. Alternatively, viral vectors can be used that provide for transient expression. Such vectors can be repeatedly administered as necessary. Delivery of vectors encoding the subject scaffold therapeutic agent can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

SEQUENCES

EphB4 precursor
SEQ ID NO: 1

| | |
|---|---|
| 1 | melrvllcwa slaaaleetl lntkletadl kwvtfpqvdg qweelsglde eqhsvrtyev |
| 61 | cdvqrapgqa hwlrtgwvpr rgavhvyatl rftmleclsl pragrscket ftvfyyesda |
| 121 | dtataltpaw menpyikvdt vaaehltrkr pgaeatgkvn vktlrlgpls kagfylafqd |
| 181 | qgacmallsl hlfykkcaql tvnltrfpet vprelvvpva gscvvdavpa pgpspslycr |
| 241 | edgqwaeqpv tgcscapgfe aaegntkcra caqgtfkpls gegscqpcpa nshsntigsa |
| 301 | vcqcrvgyfr artdprgapc ttppsaprsv vsrlngsslh lewsaplesg gredltyalr |
| 361 | crecrpggsc spcggdltfd pgprdlvepw vvvrglrpdf tytfevtaln gvsslatgpv |
| 421 | pfepvnvttd revppavsdi rvtrsspssl slawavprap sgavldyevk yhekgaegps |
| 481 | svrfiktsen rselrglkrg asylvqvrar seagygpfgq ehhsqtqlde segwreqlal |
| 541 | iagtavvgvv lvlvvivvav lclrkqsngr eaeysdkhgq ylighgtkvy idpftyedpn |
| 601 | eavrefakei dvsyvkieev igagefgevc rgrlkapgkk escvaiktlk gqyterqrre |
| 661 | flseasimgq fehpniirle gvvtnsmpvm iltefmenga ldsflrlndg qftviqlvgm |
| 721 | lrgiasgmry laemsyvhrd laarnilvns nlvckvsdfg lsrfleenss dptytsslgg |
| 781 | kipirwtape aiafrkftsa sdawaygivm wevmsfgerp ywdmsnqdvi naieqdyrlp |
| 841 | pppdcptslh qlmldcwqkd rnarprfpqv vsaldkmirn paslkivare nggashplid |
| 901 | qrqphysafg svgewlraik mgryeesfaa agfgsfelvs qisaedllri gvtlaghqkk |
| 961 | ilasvqhmks qakpgtpggt ggpapqy |

Ephrin B2
SEQ ID NO: 2

| | |
|---|---|
| 1 | mavrrdsvwk ycwgvlmvlc rtaisksivl epiywnssns kflpgqglvl ypqigdkldi |
| 61 | icpkvdsktv gqyeyykvym vdkdqadrct ikkentplln cakpdqdikf tikfqefspn |
| 121 | lwglefqknk dyyiistsng slegldnqeg gvcqtramki lmkvgqdass agstrnkdpt |
| 181 | rrpeleagtn grssttspfv kpnpgsstdg nsaghsgnni lgsevalfag iasgciifiv |
| 241 | iiitlvvlll kyrrrhrkhs pqhtttlsls tlatpkrsgn nngsepsdii iplrtadsvf |
| 301 | cphyekvsgd yghpvyivqe mppqspaniy ykv |

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to these skilled in the art upon review of this specification and the claims below. The full scope of the invention should be determined by reference to the claims, along with their full scope of equivalents, and the specification, along with such variations.

1-52. (canceled)

53. An isolated non-immunoglobulin antigen binding scaffold comprising an antigen binding domain that binds to an epitope situated in the extracellular portion of EphB4 or Ephrin B2 and inhibits an EphB4 or Ephrin B2 activity.

54. The isolated non-immunoglobulin antigen binding scaffold of claim 53, wherein the non-immunoglobulin antigen binding scaffold inhibits vascularization of a tissue in vivo.

55. The isolated non-immunoglobulin antigen binding scaffold of claim 53, wherein the non-immunoglobulin antigen binding scaffold binds an epitope selected from amino acids 16-198 of the EphB4 sequence, amino acids 327-427 of the EphB4 sequence, and amino acids 428-537 of the EphB4 sequence.

56. An isolated non-immunoglobulin antigen binding scaffold comprising an antigen binding domain of claim 53, wherein the isolated non-immunoglobulin antigen binding scaffold is covalently linked to an additional functional moiety.

57. The isolated non-immunoglobulin antigen binding scaffold of claim 56, wherein the additional functional moiety confers increased serum half-life on the non-immunoglobulin antigen binding scaffold comprising an antigen binding domain.

58. The isolated non-immunoglobulin antigen binding scaffold of claim 56, wherein the additional functional moiety is a label.

59. The isolated non-immunoglobulin antigen binding scaffold of claim 53, wherein the non-immunoglobulin antigen binding scaffold is selected from an antibody substructure, a minibody, an adnectin, an anticalin, an affibody, a knottin, a glubody, a C-type lectin-like domain protein, a tetranectin, a kunitz domain protein, a thioredoxin, a cytochrome b562, a zinc finger scaffold, a Staphylococcal nuclease scaffold, a fibronectin or a fibronectin dimer, a tenascin, an N-cadherin, an E-cadherin, an ICAM, a titin, a GCSF-receptor, a cytokine receptor, a glycosidase inhibitor, an antibiotic chromoprotein, a myelin membrane adhesion molecule P0, a CD8, a CD4, a CD2, a class I MHC, T-cell antigen receptor, a CD1, a C2 and I-set domains of VCAM-1, a 1-set immunoglobulin domain of myosin-binding protein C, a 1-set immunoglobulin domain of myosin-binding protein H, a I-set immunoglobulin domain of telokin, an NCAM, a twitchin, a neuroglian, a growth hormone receptor, an erythropoietin receptor, a prolactin receptor, an interferon-gamma receptor, a β-galactosidase/glucuronidase, a β-glucuronidase, a transglutaminase, a T-cell antigen receptor, a superoxide dismutase, a tissue factor domain, a cytochrome F, a green fluorescent protein, a GroEL, and a thaumatin.

60. The isolated non-immunoglobulin antigen binding scaffold of claim 53, wherein the non-immunoglobulin antigen binding scaffold inhibits the EphrinB2-stimulated autophosphorylation of EphB4.

61. The isolated non-immunoglobulin antigen binding scaffold of claim 53, wherein the non-immunoglobulin antigen binding scaffold inhibits the binding of EphrinB2 to the extracellular portion of EphB4.

62. The isolated non-immunoglobulin antigen binding scaffold of claim 53, wherein the epitope is the first fibronectin-like domain (FND1) of EphB4.

63. The isolated non-immunoglobulin antigen binding scaffold of claim 53, wherein the non-immunoglobulin antigen binding scaffold is clinically acceptable for administration to a human.

64. A pharmaceutical preparation comprising the isolated non-immunoglobulin antigen binding scaffold of claim 53.

65. The pharmaceutical preparation of claim 64 for treating cancer.

66. Use of an isolated non-immunoglobulin antigen binding scaffold comprising an antigen binding domain of claim 53 to make a pharmaceutical preparation for treating cancer.

67. A method of treating cancer, the method comprising administering to a patient in need thereof an effective amount of an isolated non-immunoglobulin antigen binding scaffold comprising an antigen binding domain that binds to an epitope situated in the extracellular portion of EphB4 or Ephrin B2 and inhibits an EphB4 or Ephrin B2 activity.

68. The method of claim 67, wherein the patient is diagnosed with a cancer selected from colon carcinoma, breast tumor, mesothelioma, prostate tumor, squamous cell carcinoma, Kaposi sarcoma, and leukemia.

69. The method of claim 67, wherein the isolated non-immunoglobulin antigen binding scaffold is administered systemically or locally.

70. A method of inhibiting angiogenesis in a patient, the method comprising administering to a patient in need thereof an effective amount of an isolated non-immunoglobulin antigen binding scaffold comprising an antigen binding domain that binds to an epitope situated in the extracellular portion of EphB4 or Ephrin B2 and inhibits an EphB4 or Ephrin B2 activity.

71. The method of claim 70, wherein the patient is diagnosed with macular degeneration.

72. The method of claim 70, wherein the isolated non-immunoglobulin antigen binding scaffold is administered systemically or locally.
